(19) United States
(12) Patent Application Publication
(10) Pub. No.: US 2010/0098755 A1
(43) Pub. Date: Apr. 22, 2010

(54) PROCESS OF PRODUCING DIRECTLY COMPRESSED TABLETS OF STEROLS AND/OR STANOLS

(75) Inventor: Daniel Debeyer, Vancouver (CA)

Correspondence Address: KIRTON AND MCCONKIE, 60 EAST SOUTH TEMPLE,, SUITE 1800, SALT LAKE CITY, UT, 84111, US

(73) Assignee: FORBES MEDI-TECH INC., Vancouver, BC (CA)

(21) Appl. No.: 12/255,814

(22) Filed: Oct. 22, 2008

(51) Int. Cl. A61K 9/20; A61K 31/56

(52) U.S. Cl. 424/464; 514/170; 514/182

(57) ABSTRACT

A method of producing a tablet which comprises sterols and stanols includes the steps of forming the sterols and/or stanols, separately or together, into spherically-shaped, substantially uniform beads or prills and directly compressing the beads or prills into a tablet core.

### FIELD OF THE INVENTION

This present invention relates to the field of tablets comprising specific naturally derived hydrophobic compounds, particularly sterols and stanols and the means by which such sterols or stanols can be readily incorporated, by direct compression, into tablet form for nutraceutical and pharmaceutical usage.

### BACKGROUND OF THE INVENTION

While recent advances in science and technology are helping to improve quality and add years to human life, the prevention of atherosclerosis, the underlying cause of cardiovascular disease (“CVD”) has not been sufficiently addressed. Atherosclerosis is a degenerative process resulting from aninterplay of inherited (genetic) factors and environmental factors such as diet and lifestyle. Research to date suggest that cholesterol may play a role in atherosclerosis by forming atherosclerotic plaques in blood vessels, ultimately cutting off blood supply to the heart muscle or alternatively to the brain or limbs, depending on the location of the plaque in the arterial tree¹,². Data from the early Framingham Epidemiological Study indicates that increases in serum cholesterol levels are associated with increased risk of death from CVD³. More recent studies confirm that CVD is a leading cause of death and disability in industrialized nations⁴.

Studies have indicated that a 1% reduction in a person's total serum cholesterol yields a 2% reduction in risk of a coronary artery event⁵. Statistically, a 10% decrease in average serum cholesterol (e.g. from 6.0 mmol/L to 5.3 mmol/L) may result in the prevention of 100,000 deaths in the United States annually⁶.

As the population becomes increasingly aware of the importance of maintaining cholesterol balance in check, the need for naturally derived, safe and effective agents which address the underlying causes of CVD, and which can be readily incorporated into a wide variety of delivery means, becomes even more apparent.

One focus of such research related to naturally derived, safe and effective agents to address the underlying causes of CVD has been plant-derived sterols and stanols (also known as phytosterols and phytostanols). Sterols are naturally occurring compounds that perform many critical cellular functions. Phytosterols such as campesterol, stigmasterol and beta-sitosterol in plants, ergosterol in fungi and cholesterol in animals are each primary components of cellular and sub-cellular membranes in their respective cell types. The dietary source of phytosterols in humans comes from plant materials i.e. vegetables and plant oils. The estimated daily phytosterol content in the conventional western-type diet is approximately 60-80 milligrams in contrast to a vegetarian diet which would provide about 500 milligrams per day.

Phytosterols have received a great deal of attention due to their ability to decrease serum cholesterol levels when fed to a number of mammalian species, including humans. While the precise mechanism of action remains largely unknown, the relationship between cholesterol and phytosterols is apparently due in part to the similarities between the respective chemical structures (the differences occurring in the side chains of the molecules). It is assumed that phytosterols displace cholesterol from the micellar phase and thereby reduce its absorption or possibly compete with receptor and/or carrier sites in the cholesterol absorption process.

Over forty years ago, Eli Lilly marketed a sterol preparation from tall oil and later from soybean oil called Cytellin™ which was found to lower serum cholesterol by about 9% according to one report.⁷ Various subsequent researchers have explored the effects of sitosterol preparations on plasma lipid and lipoprotein concentrations⁸ and the effects of sitosterol and campesterol from soybean and tall oil sources on serum cholesterols.⁹ Compositions have been explored in which phytosterols or phytostanols (their hydrogenated counterparts) are esterified in order to enhance solubility. One composition of phytosterols which has been found to be highly effective in lowering serum cholesterol is disclosed in U.S. Pat. Ser. No. 5,770,749 to Kutney et al.

Despite the obvious and now well recorded advantages of phytosterols, not only in the treatment of CVD and its underlying conditions such as hypercholesterolemia, hyperlipidemia, atherosclerosis, hypertension, thrombosis but in the treatment of other diseases such as Type II diabetes, dementia cancer and aging, the administration of phytosterols and the incorporation thereof into tablets and other delivery vehicles has been complicated by the fact that they are highly sticky and waxy, when extracted from the source. This makes their flow and processing into delivery vehicles, such as foods and nutraceuticals, very difficult.

In the food area, early research on phytosterols focused on grinding or milling the phytosterols in order to enhance their solubility (U.S. Pat. Serial Nos: 3,881,005 and 4,195,084 both to Eli Lilly). In addition, researchers have looked to the esterification of phytosterols in order to enhance their solubility. German Patent 2035069/Jan. 28, 1971 (analogous to U.S. Pat. No. 3,751,569) describes the addition of phytosterol fatty acid esters to cooking oil. The esterification is carried out between a free sterol and a fatty acid anhydride, with perchloric acid as the catalyst. The significant drawback to this process, along with others, is the use of non-food grade catalysts and reagents.

There are unique issues and problems in regards to tableting sterols and stanols as they are inherently waxy materials which are hydrophobic, typically do not flow well in micronized form and have a low bulk density. These properties cause problems during the tablet making (tableting) process, which include but are not limited to, picking and sticking of materials to tooling, materials sticking to the press turntable during compression and poor tablet weight control. Further, sterols are difficult to mill because they tends to clog the mill screen unless cryo-milled. Prior to the present invention, tablets high in sterols typically exhibited poor compressibility and once compressed had slow tablet disintegration adversely impacting delivery of the phytosterol upon ingestion. The timely disintegration of sterols/stanols is important due to their mode of efficacy, in particle form, in the gastrointestinal lumen.

In view of the technical difficulties involved in adding phytosterols to foods and beverages, and bearing in mind the utility of being able to widely supplement a wide variety of comestible products with these components, it would be highly advantageous to find an effective means of dispersing or suspending phytosterols in aqueous media at high concentrations or in creating a means to deliver phytosterols in a manner which addresses the problems of waxiness and guminess attendant in the powder formulation, thereby opening up the possibility of providing low fat or fat-free products (i.e. aqueous based products) containing phytosterols in a variety of formats.

It is an object of the present invention to obviate or mitigate the above noted disadvantages and to find a solution for the problem plaguing manufacturers wishing to widely use hydrophobic compounds, such as phytosterols, in these varied formats.

### SUMMARY OF THE INVENTION

The present invention provides a unique method of producing a Tablet comprising sterols and stanols. Specifically, the method comprises:
a) forming the sterols and/or stanols, separately or together, into spherically-shaped, substantially uniform beads or prills; and
b) directly compressing the beads or prills into a Tablet Core.

More preferably, the formation of beads at step a) is achieved by prilling, which comprises i) melting the sterols and/or stanols into a liquid composition; ii) forming the liquid composition into liquid droplets; iii) solidifying the liquid droplets to form spherically-shaped, substantially uniform beads or prills.

Optionally, at step a) one or more excipients, may be added to the individual sterol, stanol, and/or ester prior to prilling. The method can be used to manipulate the composition of the tablet by co-melting varying amounts of sterols and/or stanols, for example, to achieve the exact specifications/formulations. Optionally the Tablet Core may be coated with one or more Tablet Coatings.

The present method provides a quick and simple method of producing hard tablets comprising sterols, stanols, and/or esters. In particular, prllinci and directly compressing sterols and stanols to produce hard tablets allows for a cost-efficiency in producing dietary supplements.

The present invention further provides a method for treating or preventing CVD and its underlying conditions including atherosclerosis, hypercholesterolemia, hyperlipidemia, hypertension, thrombosis, and related diseases such as Type II diabetes, as well as other diseases that include oxidative damage as part of the underlying disease process such as dementia, aging, and cancer by administering to an animal a tablet, prepared in accordance with the method of the present invention.

### PREFERRED EMBODIMENTS OF THE INVENTION

The following detailed description is provided to aid those skilled in the art in practising the invention. However this detailed description should not be construed so as to unduly limit the scope of the present invention. Modifications and variations to the embodiments discussed herein may be made by those with ordinary skill in the art without departing from the spirit or scope of the present invention.

As used herein, the term “Tablets” means items for ingestion that are intended to supplement the diet without being represented for use as a conventional food or as the sole item of a meal or diet and that meets the definition of dietary supplements as found in the United States Dietary Supplement Health and Education Act (DSHEA). Tablets are solid dosage forms usually prepared with the aid of suitable pharmaceutical excipients or diluents. Conventionally, tablets are primarily prepared by compression of powders or granules into desired shapes, sizes, designs, dosages, layers, and coatings. Compressed tablets may be made by three basic methods, namely wet granulation, dry granulation, and direct compression. Direct compression is typically the simplest method, but requires certain properties from the material being compressed in order to facilitate this tablet production method.

As used herein, the term “Tablet Coatings” means a mixture of one or more ingredients used to accomplish one or more of the following objectives: i) to protect a Tablet Core from exposure to air/humidity/ph or other influences or from chipping and breakage; ii) to mask the taste of the Tablet Core; iii) to provide special characteristics of release of the active ingredient in the Tablet Core; iv) to provide or enhance the aesthetics of the Tablet Core; v) to distinguish or differentiate the Tablet or vi) to improve the swallowability of the Tablet Core.

As used herein, the term “Tablet Core” means the entirety of the Tablet, excluding the Tablet Coating.

As used herein, the term “sterol” includes all sterols without limitation, for example: (from any source and in any form: a, D and y) sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, cholesterol and all natural or synthesized forms and derivatives thereof, including isomers.

The term “stanol” refers to, for example: (from any source and in any form: α, β and γ) saturated or hydrogenated sterols including all natural or synthesized forms and derivatives thereof, and isomers, including sitostanol, campestanol, stigmastanol, brassicastanol (including dihydrobrassicastanol), desmostanol, chalinostanol, poriferastanol, clionastanol, ergostanol, coprostanol, codistanol, isofucostanol, fucostanol, clerostanol, nervistanol, lathostanol, stellastanol, spinastanol, chondrillastanol, pepostanol, avenastanol, isoavenastanol, fecostanol, and pollinastastanol.

It is to be understood that modifications to the sterols and stanols i.e. to include side chains also falls within the purview of this invention. It is also to be understood that, when in doubt throughout the specification, and unless otherwise specified, the term “sterol” encompasses both sterol and stanol. The terms “phytosterol” and “phytostanol” may also be used and refer to all plant-derived sterols or stanols respectively.

The sterols and stanols for use in forming derivatives in accordance with this invention may be procured from a variety of natural sources or they may be artificially synthesized. For example, they may be obtained from the processing of plant oils (including aquatic plants) such as corn oil and other vegetable oils, wheat germ oil, soy extract, rice extract, rice bran, rapeseed oil, sunflower oil, sesame oil and fish (and other marine-source) oils. They may also be derived from yeasts and fungi, for example ergosterol. Accordingly, the present invention is not to be limited to any one source of sterols. U.S. Pat. Ser. No. 4,420,427 teaches the preparation of sterols from vegetable oil sludge using solvents such as methanol. Alternatively, phytosterols and phytostanols may be obtained from tall oil pitch or soap, by-products of forestry practises as described in U.S. Pat. Ser. No. 5,770,749, incorporated herein by reference. A further method of extracting sterols and stanols from tall oil pitch is described in Canadian Patent Application Serial No. 2,230,373 which was filed on Feb. 20,1998 (corresponding to PCT/CA99/00150 which was filed on Feb. 19, 1999) and U.S. patent application Ser. No. 10/060,022 which was filed on Jan. 28, 2002 the contents of all of which are incorporated herein by reference.

Accordingly, it is to be understood that the widest possible definition is to be accorded to the terms “sterol” and “stanol” as used herein, including, but not limited to: free sterols and stanols, esterified sterols and stanols with aliphatic or aromatic acids (thereby forming aliphatic or aromatic esters, respectively), phenolic acid esters, cinnamate esters, ferulate esters, phytosterol and phytostanol glycosides and acylated glycosides or acylglycosides. Thus, the terms “sterols” and “stanols” encompasses all analogues, which may further have a double bond at the 5-position in the cyclic unit as in most natural sterols, or one or more double bonds at other positions in the rings (for example, 6, 7, 8(9), 8(14), 14 5/7) or no double bonds in the cyclic unit as in stanols. Further, there may be additional methyl groups as, for example, in α₁-sitosterol.

Phytosterols and/or phytostanols once isolated from their source are generally formed into a solid powder through precipitation, filtration and drying, spray drying, lyophilization or by other conventional work-up techniques. It is this powder form which has, heretofore, provided many of the challenges in incorporating phytosterols into hard tablets. Within the scope of the present invention, it has been surprisingly found that by applying prilling technology to sterols and stanols, the present inventors have overcome significant challenges in tableting of these products.

Tablets are solid dosage forms usually prepared with the aid of suitable pharmaceutical excipients. They may vary in size, shape, weight, hardness, thickness, disintegration, and dissolution characteristics and in other aspects, depending on their intended use and method of manufacture. Most tablets are used in the oral administration of drugs. Many of these are prepared with colourants and coatings of various types.

Tablets are prepared primarily by compression. Compressed tablets are manufactured with tablet machines capable of exerting great pressure in compacting the powdered or granulated material. Their shape and dimensions are determined by use of various shaped punches and dies.

The physical features of compressed tablets are well known, and include, without limitation, round, oblong, or unique in shape; thick or thin; large or small in size; flat or convex; unscored or scored; engraved or imprinted; coated or uncoated; coloured or uncoloured; one, two, or three layered. Compressed tablets may be made by three basic methods: wet granulation, dry granulation, and direct compression.

Wet granulation involves the steps of (a) weighing and blending the ingredients, (b) preparing a damp mass, (c) screening the damp mass into pellets or granules, (d) drying the granulation, (e) sizing the granulations by dry screening, (f) adding lubricant and blending, and (g) forming tablets by compression.

Dry granulation involves the step of compacting a powder mixture in large pieces and subsequently breaking down the pieces into granules. A diluent with cohesive properties is typically added.

For materials that already possess free-flowing and cohesive properties, direct compression is possible, and is the simplest method of tablet production. Any direct compression method and/or apparatus for producing tablets is contemplated by the present invention.

Most powdered medicinal agents require addition of excipients such as diluents, binders, disintegrants, and lubricants to provide the desired characteristics for tablet manufacture and efficacious use. One important requirement in tablet manufacture is that the drug mixture flow freely from the hopper of the tablet press into the dies to enable high-speed compression of the powder mix into tablets.

It has been discovered in the present invention that prilling of sterols/stanols also provides for material that is desirably free flowing, yet cohesive, to allow for easy direct compression into hard tablets. Prior to the application of prilling technology to sterols and stanols, it was necessary to granulate them before direct compression.

So, as used herein, the term prilling is a technique known in the art for producing spheres or particles of a substance. Generally, prilling involves the melting of a substance into a liquid, passing or forcing the liquid through a hole or holes of desired size to produce liquid drops, and cooling the liquid drops to produce solid spheres or particles of said substance.

Prilling is also known as melt spraying, spray congealing, spray chilling, or melt atomization. The process involves taking molten liquids or mixtures, atomizing these liquids and cooling the resultant droplets to form a prill or bead.

Prilling is a relatively cheap process for the conversion of molten materials into a solid, easy to handle form. While the final form can vary depending on the melt viscosity from fibers to rods to spheres, the typical output is spheres or beads and such is desired within the scope of the present invention. A wide range of final sizes of spheres or beads are possible depending on the atomization method and the starting material. An average particle size distribution of 10-3,000 microns is readily obtainable. Throughputs of 1-2,000 lbs./hour and higher are easy to obtain with the above size distributions. The process can be used with blended materials very easily and as such desired ratios and combinations of sterols and stanols can be co-blended during the prilling process and prior to direct tablet compression.

It is important to understand that the present inventors are not claiming that there is novelty or inventiveness in prilling sterols and stanols per se. However, what is novel and inventive are the following:
1) A method of direct compression of spherical prills or beads comprising sterols and/or stanols into tablets.
2) A method of co-blending desired ratios and combinations of sterols and stanols during the prilling process and prior to direct compression of the spherical prills or beads comprising sterols and/or stanols into tablets.
3) A method of co-blending other desired therapeutic aids or excipients with the sterols and stanols during the prilling process and prior to the direct compression of spherical prills or beads comprising sterols and/or stanols into tablets.
4) The use of these tablets, so formed in accordance with 1) to 3) by way of administration to animals, including humans, for the treatment and/or prevention of CVD and its underlying conditions including atherosclerosis, hypercholesterolemia, hyperlipidemia, hypertension, thrombosis, and related diseases such as Type II diabetes, as well as other diseases that include oxidative damage as part of the underlying disease process such as dementia, aging, and cancer.

Any of the conventionally known prilling methods and/or apparatus for producing prills may be used in accordance with the present invention with such prills then being used to fulfill the entire purpose of the present invention.

WO0137681 to Gottemoller discloses the general prilling of phytosterols, as seen in the following quote taken from the first paragraph of page 10 : “In a preferred embodiment, the phytosterols or phytostanols are ground or prilled to produce a powdered product before they are added to the aqueous mixture. Prilling is a well known process, and any prilling process known in the art may be used in the present invention. See, e.g., U.S. Pat. No. 4,238,429. Preferably, the phytosterols orphytostanols are spray prilled. Grinding or prilling the phytosterols or phytostanols prior to their addition to the aqueous mixture allows for a free-flowing product, which helps incorporate the compounds into the aqueous system.” The entire contents of Gottemoller are incorporated herein by reference.

However, it must be understood that Gottemoller does not recognize the usefulness, ease of handling, cost-efficiency, and ease of use of these prills when directly compressed into dietary supplement phytosterol tablets.

US2006/0024352 to Poxon et al. discloses the optional use of “a non-micronized particle size/prilled phytosterol and or phytostanol” to form a chewable tablet only because mastication during consumption of chewable tablets results in particle size reduction. Poxon et al. do not recognize the advantages of using specifically prilled phytosterols/stanols to form hard types of tablets, those advantages being flowability, ease of handling and use, and relatively simple and inexpensive direct compression of the prilled product as opposed to the more expensive and complex granulation methods focussed on by Poxon et al.

EP0925293 to Walsh et al teaches a process of incompletely melting a composition comprising phytosterols, and subsequently atomizing the incompletely melted mass. It is disclosed that the resulting powder may be used in producing phytosterol tablets. However, it is critical to note that Walsh et al actually teaches away from the advantages of fully melting the phytosterols into a homogenous molten mixture and fully prilling the phytosterols seen in the present invention, and, indeed, Walsh et al emphasizes the critical importance in their invention of not fully melting their composition.

U.S. Pat. No. 6,911,164 (20030057579). Cognis. Granted Jun. 28, 2005. Equivalent to European Patent 1297756. Granted Feb. 9, 2005 teaches the prilling of phytosterols by way of a two-stage process for the solidification and cooling of droplets:.

In the first stage, the surface of the spheres is solidified by quenching with liquid, evaporating medium. In the second stage, residual solidification and further cooling take place in a final cooling unit.

Droplet formation can also be carried out using a casting plate and vibrating membrane of the type marketed, for example, by the firm GMF Gouda under the name of “Jet Priller”.

The sterol melt is passed by a controlled excess pressure of from about 300 to about 2,000 mbar through one or more droplet forming systems which disperse the continuous product stream into droplets with a diameter of from about 1.6 to about 1.9 times the perforation diameter. The shape of the droplets is stabilized by cryogenic countercurrent cooling. i.e. by spraying in liquid evaporating nitrogen or carbon dioxide in a special prill solidifying tube (dropping height from about 1 to abouut 10 m, preferably from about 1 to about 2 m).

Dissipation of the residual heat of crystallization and final cooling and dissipation of the residual heat for complete crystallization take place in a rotating fluidized bed with cooled gas which is preferably circulated and which, preferably, is or may be the same gas as used for quenching. It is also cooled in the fluidized bed to the product discharge temperature.

The entire contents of U.S. Pat. No. 6,911,164 are incorporated herein by reference.

The principle steps behind the prilling process is the melt of the sterol composition followed by the exposure of the molten sterols to a drop forming system and exposing the droplets to a cooling medium flowing in countercurrent to them or contacting the droplets with a cooling medium which solidifies and cools them. The temperature at which the sterols are introduced into the droplet forming tower is limited by the solidification range of the sterol preparations and, according to the invention, is in the range from about 110 to about 170° C., preferably in the range from about 110 to about 150° C. and more particularly in the range from about 130 to about 140° C. Feed temperatures of from about 5 to about 40° C. and preferably from about 10 to about 25° C. above the solidification point of the sterols have proved to be particularly effective. At these temperatures, the viscosity of the melt is in such a range that the sterol preparations can already be readily converted into droplets

In a preferred form, the sterol preparations are introduced into a prilling tower or apparatus of similar function as a melt via an atomization nozzle. There are a number of means to achieve such atomization, including using single fluid nozzle, two-fluid nozzles, rotary or spinning discs, perforated discs or the like. The capacity of such perforated disks, which normally have from about 10 to about 750 perforations, is preferably in the range from about 0.3 to about 6 kg/h/perforation for a perforation diameter of from about 0.15 to about 1.2 mm. The droplets obtained have a diameter of from about 1.55 to about 2.0 times the diameter of the perforations.

Beyond the atomisation device, the most important part is a process chamber to confine the droplets and the process gas. This chamber must have sufficient—but minimal—dimensions and still envelope the flight paths of the droplets,—and even the biggest and furthest flying particles of the distribution must not hit the walls before it is safe to do so.

The droplets fall vertically downwards through the prilling/dropping tower in the substantial absence of turbulence. Although cooling can be carried out with a cold liquid (for example droplet formation in water) or a cold evaporating liquid (evaporation of liquid nitrogen or carbon dioxide), cooling in the dropping tower with a cold gas flowing in countercurrent—as adequately described in prior art—is recommended for practical reasons. The cooling gas is supplied as a cooled fresh gas or a cooled recirculated gas. Besides air, inert gases such as, for example, nitrogen or carbon dioxide may of course also be used as the cooling gas.

A particular feature of this process is that cooling to the solidification temperature, i.e. solidification and subsequent cooling to the discharge temperature, takes place in a long dropping tower with cold gas as a direct heat transfer medium.

In preferred embodiments of the present invention, and following the teachings of these the prilling process parameters described herein by way of example and also following known prilling processes, in one preferred embodiment, sterol/stanol prills of under 30 microns in diameter are produced. In another preferred embodiment, prills of over 200 microns in diameter are produced.

Although not required, under some circumstances it may be desirable to melt other adjuncts and/or components to co-prill with the sterols using the process described herein and prior to direct compression tableting of the prills. Such adjuncts include, but are not limited to: diluents, fillers, binders, coatings, adhesives, disintegrants, anti-adherents, glidants, lubricants, colourants and flavourants.

Furthermore, in another embodiment of the present invention, it may be desirable to select particular types and/or ratios and sterols and stanols, melt each and then co-introduce these components in the prilling tower with the resultant prilled product having a desired composition of sterols and/or stanols.

After formation of the spherically-shaped, substantially uniform beads orprills, they are directly compressed into a Tablet Core. This Tablet Core may be administered as is or may be further coated with one or more Tablet Coatings. Generally, the sterol prills (along with one or more excipients, fillers, binders, adhesives, colourants, flavourants or disintegrating agents as and if desired) are placed in a die for compression in the desired dosage shape and size. Of importance to note is that sterol prills are directly compressible and need not be subject to wet or dry granulation prior to compression.

This particular advantage has not heretofor been appreciated. Direct compression is the preferred technique since it is considered that fewer chemical stabilty problems are associated with this technique in comnparison with the wet granulaton process as rnoisture is considered to be a primary cause of instabiity in tablet dosage torms. In addition to the advantage of rnproved active ingredient stability, the use of direct compression makes it unnecessary to use applied heat to dry the damp granule. Other benefits associted with direct compression are related to particle size uniformity.

Generally, the process of direct compression is a process of applying pressure (via an upper and a lower punch) to materials held in a die cavity. The events that occur in the process of compression are (1) transitional repacking, (2) deformation at point of contact, (3) fragmentation and/or deformation, (4) bonding, (5) deformation of the solid body, (6) decompression, and (7) ejection.

The following examples are meant to help illustrate, but not limit, the present invention.

### EXAMPLES

### Example 1

Prilling of Phytosterol Parameters (ReducolTM sterol blend is a unique proprietary blend comprising primarily beta-sitosterol, sitostanol, campesterol and campestanol)
1. Reducol™ Properties
   a. Wood phytosterols fortified with wood phytostanols. Major component, sitosterol.
   b. High melt temperature (melting point of about 145 C/295 F.
   c. Product is waxy and sticky at room temperature. Powder has tendency to clump in box.
2. General System Requirements
   a. Avoid excessive contact surface temperature.
   b. Ensure all systems rated to operating temperatures up to 175 C/350 F.
3. Melt System
   a. Reducol™ has a melting temperature of about 145 C (295 F).
   b. To facilitate product melting, suggest a higher melt temperature of 310-320 F. Unacceptably long melt times observed when melting at 145 C.
   c. Product can char if exposed to high temperature. Recommend maximum contact surface temperature of 175 C (350 F). Charring temperature presently not known.
   d. Significant potential for product caking onto tank walls and mixing surfaces.
   e. Maintain a nitrogen blanket within system at all times.
4. Transfer Line
   a. Maintain system above 310 F until recirculation system operating well.
   b. Avoid temperature hotspots within system (maximum contact surface temperature of 175 C (350 F).
   c. Valves and gauges may not operate properly due to product buildup within system.
5. Spray Nozzle System
   a. Suggest using hollow-core pressure nozzle, single fluid.
   b. Suggested pressure 30-40 psi. Refer to target particle size and adjust spray system accordingly.
   c. Potential for high proportion of overs (greater than 20 mesh).
6. Prilling Tower
   a. Given the sticky nature of ReducolTM, potential for product buildup within tower. Until sufficient stability on the product is know, retain but do not re-melt the recovered product.
7. Powder Conveying
   a. Reducol™ is prone to stick to all surfaces. Surfaces with tight tolerances may experience product buildup and sticking.
   b. Conveyor type system may be more effective than cyclone type system.
8. Product Packaging
   a. Screen product through 16 mesh screen prior to packaging.
   b. Reducol™ is sticky and will frequently blind screens (even 16 mesh). Recommend frequent monitoring of the performance of the screen.
   c. Retain screen overs for reprocessing at a later date.
   d. Package product into PE lined boxes of 20 kg. Screen boxes through metal detector.

### Target ReducoI™ Particle Size

Laser Light Scattering
Median, 350 microns
D(10), 175 micron
D (90), 550 micron

Rotap Analysis (100 grams, 10 minutes of taping)

| Mesh | % Retained on Screen |
|---|---|
| 14 | <0.3 |
| 20 | 4 |
| 30 | 20 |
| 40 | 35 |
| 60 | 30 |
| 80 | 8 |
| 100 | 2 |
| Pan | <2 |

### Example 2

### Direct Compression Tabletting of Prilled Phytosterol

Formulation Composition

| | | Reducol TB-900-1 | | Reducol TB-900-2 | |
|---|---|---|---|---|---|
| Component | Purpose | MG/TAB | KG/BATCH | MG/TAB | KG/BATCH |
| Reducol m300 | Active | 918 | 32.4 | 918 | 27.5 |
| Microcrystalline | Diluent | 518 | 18.3 | 733.6 | 22.0 |
| Cellulose | | | | | |
| DiCalcium | Diluent | 214 | 7.55 | 304.4 | 9.13 |
| Phosphate | | | | | |
| Silicon Dioxide | Glidant | | | 20 | 0.6 |
| Croscarmellose | Disintegrant | 34 | 1.20 | | |
| Sodium | | | | | |
| Magnesium | Lubricant | 8.5 | 0.3 | 10 | 0.3 |
| Stearate | | | | | |
| Stearic Acid | Lubricant | 25.50 | 0.9 | 32 | 0.96 |
| White Coating | Coating | | 1.21 | | 1.21 |
| Total | | 1718 | 61.81 | 2018 | 61.71 |

Manufacturing Process (Batch Data in Bold)
1. Screened all components through 15-20 mesh screen (16 mesh for Reducol, all others 20 mesh).
2. Combined all components except lubricants in suitably sized V-blender (4.5 Cubic Foot). Blended for appropriate length of time (4 minutes).
3. Added lubricants to V-blender and blend for appropriate length of time (2 minutes).
4. Discharged blend through 10 mesh screen into drum.
5. Set up tablet press with tablet tooling. (Fette 2090 tablet press, D-tooling, 0.3880×0.8750″ modified oval with bisect).
6. Optimized tablet press run conditions. Typical conditions

| | Reducol | Reducol |
|---|---|---|
| Parameter | TB 900-1 | TB 900-2 |
| Hardness (Kp) | 30-34 | 40-44 |
| Run Speed (press | 85,000 | 85,000 |
| output/hr) | | |
| Precompression (KN) | 10 | 9.6 |
| Main compression | 10.3 | 9.1 |

7. Prepared coating solution by mixing coating with water in suitable container.
8. Placed bulk tablets into coating pan and spray coated solution on tablets with heating and pan rotation. Continued spraying until coating uniformly applied to tablets with a weight gain of 2.5-3 percent. Actual weight gain on lab scale of:
   a. TB 900-1=2.66%
   b. TB 900-2=2.56%
9. Released test tablets

### REFERENCES

1. Law M. R., Wald N. J., Wu., Hacksaw Z A., Bailey A.; Systemic underestimation of association between serum cholesterol concentration and ischemic heart disease in observational studies: Data from BUPA Study; Br. Med. J. 1994; 308:363-366

2. Law M. R., Wald N. J., Thompson S. G.; By how much and how quickly does reduction in serum cholesterol concentration lower risk of ischemic heart disease? Br. Med. J. 1994; 308:307-373

3. Kannel W B, Castelli W P, Gordon T et al. Lipoprotein cholesterol in the prediction of atherosclerotic disease: new perspectives based on the Framingham Heart Study. Ann Intern Med. 1995;90:85-91

4. Singh B K, Mehta J L. Management of dyslipidemia in the primary prevention of coronary heart disease. Curr Opin Cardiol. 2002; 17:503-11

5. La Rosa J. C., Hunninghake D., Bush D. et al.; The cholesterol facts: A summary of the evidence relating to dietary fats, serum cholesterol and coronary heart disease:A joint statement by the American Heart Association and the National Heart, Lung and Blood Institute. Circulation 1990; 81:1721-1733

6. Havel R. J., Rapaport E., Drug Therapy: Management of Primary Hyperlipidemia. New England Journal of Medicine, 1995; 332:1491-1498

7. Kuccodkar et al.; Effects of plant sterols on cholesterol metabolism. Atherosclerosis, 1976; 23:239-248

8. Lees R. S., Lees A. M. Effects of sitosterol therapy on plasma lipid and lipoprotein concentrations. In: Greten H (Ed) Lipoprotein Metabolism. Springer-Verlag, Berlin, Heidelberg, New York, 1976:119-124

9. Lees A. M., Mok H. Y. I., Lees R. S., McCluskey M. A., Grundy S. M. Plant sterols as cholesterol-lowering agents: clinical trials in patients with hypercholesterolemia and studies of sterol balance. Atherosclerosis 1977; 28: 325-338

We claim:

1. A method of producing a tablet comprising one or more sterols or stanols or combinations thereof which comprises:
(a) forming the sterols and/or stanols, separately or together, into spherically-shaped, substantially uniform beads or prills; and
(b) directly compressing the beads or prills into a tablet.

2. The method of claim 1 wherein the sterol is selected from the group consisting of sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, and pollinastasterol.

3. The method of claim 1 wherein the stanol is selected from the group consisting of selected from the group consisting of sitostanol, campestanol, stigmastanol, brassicastanol (including dihydrobrassicastanol), desmostanol, chalinostanol, poriferastanol, clionastanol, ergostanol, coprostanol, codistanol, isofucostanol, fucostanol, clerostanol, nervistanol, lathostanol, stellastanol, spinastanol, chondrillastanol, pepostanol, avenastanol, isoavenastanol, fecostanol, and pollinastastanol

4. The method of claim 1 wherein the formation of beads at step a) is achieved by prilling, which comprises i) melting the sterols and/or stanols into a liquid composition; ii) forming the liquid composition into liquid droplets; iii) solidifying the liquid droplets to form spherically-shaped, substantially uniform beads or prills.

5. The method of claim 1 wherein, at step a), additional the prilling involves the separate prilling of each individual sterol, stanol, and/or ester thereof, optionally with one or more excipients, after which the individual sterol, stanol, and/or ester solid prills are blended together in desired amounts to produce the prilled product.

6. The method of claim 4 wherein the prilled product comprising a blend of more than one sterol, stanol, and/or ester thereof is directly compressed, optionally with one or more excipients, to produce a hard tablet comprising said blend of more than one sterol, stanol, and/or ester thereof.

7. The method of claim 5 wherein the prilled product comprising a blend of more than one sterol, stanol, and/or ester thereof is directly compressed, optionally with one or more excipients, to produce a hard tablet comprising said blend of more than one sterol, stanol, and/or ester thereof.

8. The method of claim 4 wherein the liquid droplets are solidified into spherically-shaped sterol preparations by cooling the liquid sterol droplets.

9. The method of claim 8 wherein the liquid droplets are cooled using a cooling gaseous or liquid medium.

10. The method of claim 9 wherein the cooling gaseous or liquid medium is selected from the group consisting of air, nitrogen, carbon dioxide, and mixtures thereof.

11. The method of claim 1 wherein the liquid composition of step (a) has a temperature of from about 110 to about 150° C.

12. The method of claim 1 wherein the sterols and/or stanols are formed into spherically-shaped, substantially uniform beads or prills with one or more formulation adjuncts.

13. The method of claim 12 wherein adjuncts are selected from the group consisting of diluents, fillers, binders, adhesives, disintegrants, anti-adherents, glidants, lubricants, colourants and flavourants.

14. A tablet comprising one or more sterols and stanols, or combination thereof produced by claim 1.

15. A compressed tablet for administration to humans comprising prilled sterols and/or stanols.

16. A method for treating or preventing CVD and its underlying conditions including atherosclerosis, hypercholesterolemia, hyperlipidemia, hypertension, thrombosis, and related diseases such as Type II diabetes, as well as other diseases that include oxidative damage as part of the underlying disease process such as dementia, aging, and cancer by administering to an animal a tablet, prepared in accordance with the method of claim 1.
